(19) United States
(12) Patent Application Publication
(10) Pub. No.: US 2023/0330386 A1
(43) Pub. Date: Oct. 19, 2023

(54) LIGHT THERAPY GLASSES

(71) Applicant: Texas Woman’s University, Denton, TX (US)

(72) Inventors: Brandon Rhett Rigby, Denton, TX (US); Hunter Alvis, Denton, TX (US); Brittany Rust, Denton, TX (US); Natalie Wilkinson, Cypress, TX (US)

(21) Appl. No.: 18/135,839

(22) Filed: Apr. 18, 2023

Related U.S. Application Data
(60) Provisional application No. 63/346,479, filed on May 27, 2022.
(60) Provisional application No. 63/332,246, filed on Apr. 18, 2022.

(51) Int. Cl. A61M 21/02

(52) U.S. Cl. CPC A61M 21/02; A61M 2021/0044

(57) ABSTRACT

Light therapy glasses are described herein. The glasses are a single device that is useable to provide both blue light to promote alertness in the wearer and red light that promotes sleepiness. Light is provided by blue LEDs and red LEDs positioned near but not in direct sight of the wearer’s eyes. Thus, the wearer is still able to view his/her surroundings while receiving light for their appropriate state. The glasses may include a blue light filtering lens that can be utilized in combination with the red light to further enable sleep by reducing the amount of ambient blue light being received by the wearer where the blue light may inhibit melatonin production.

### PRIORITY CLAIM

This application claims priority to U.S. Provisional Application Serial No. 63/332,246 entitled “LIGHT THERAPY GLASSES” filed Apr. 18, 2022, and to U.S. Provisional Application Serial No. 63/346,479 entitled “LIGHT THERAPY GLASSES” filed May 27, 2022, both of which are incorporated herein by reference in their entirety

### BACKGROUND

### 1. Technical Field

The present disclosure generally relates to a personal light therapy device. More specifically, the present disclosure relates to a light therapy device that provides both light that promotes a user staying awake and light that promotes the user falling asleep.

### 2. Description of Related Art

The visible light spectrum is the portion of the electromagnetic spectrum that the human eye can view. Typically, the human eye can detect wavelengths from 380-700 nm. The wavelength of light, which is related to its’ frequency and energy, determines the perceived color. The following table shows the breakdown of the visible light spectrum.

| COLOR | WAVELENGTH (nm) |
|---|---|
| Red | 625-740 |
| Orange | 590-625 |
| Yellow | 565-590 |
| Green | 520-565 |
| Cyan | 500-520 |
| Blue | 435-500 |
| Violet | 380-435 |

Blue light has shorter wavelengths, a higher frequency, and higher energy than many other colors. Most of the light emitted from the LEDs used in smartphones, TVs, and tablets contain significant levels of blue light (wavelengths between 400- 490 nm). Individuals are exposed to high-energy blue light not only through exposure to LED lighting, but naturally from sunlight. Studies have shown that blue light exposure is linked to eye strain, macular degeneration, cataracts, as well as sleep disorders. The increased use of LED digital screens/devices in recent years and the resulting increase in exposure to blue light has become more of an issue in regard to its effect on an individual’s normal sleep cycle. Blue light is considered to have the strongest effect on synchronizing/disrupting human circadian rhythm, and a range of studies have been completed/are underway trying to better understand this relationship. It is generally accepted that exposure to even low levels of blue light/bright light during night/before bedtime can disrupt the circadian rhythm/sleep cycle with resulting health implications. Blue light stimulates the brain, slowing or stopping release of the sleep inducing hormone melatonin making it harder to get a good night’s sleep. At the same time, blue light exposure during daytime is crucial for the vitality of an organism as it boosts alertness, mood, and may help memory. Studies on the effects of red light on an individual’s circadian clock have shown that this wavelength has no negative effects on the sleep cycle and promotes the secretion of melatonin, making individuals calmer, improving overall mood and mental health.

### BRIEF DESCRIPTION OF THE DRAWINGS

Advantages of the present disclosure will become apparent to those skilled in the art with the benefit of the following detailed description of embodiments and upon reference to the accompanying drawings.

FIG. 1 depicts a rear and left perspective view of light therapy glasses, according to some embodiments.

FIG. 2 depicts a front and right perspective view of light therapy glasses, according to some embodiments.

FIG. 3 depicts a front perspective view of glasses with a filter sheet attached to the glasses, according to some embodiments.

While the disclosed embodiments may be susceptible to various modifications and alternative forms, specific embodiments thereof are shown by way of example in the drawings and will herein be described in detail. The drawings may not be to scale. It should be understood, however, that the drawings and detailed description thereto are not intended to limit the present disclosure to the particular form disclosed, but to the contrary, the intention is to cover all modifications, equivalents, and alternatives falling within the spirit and scope of the present disclosure as defined by the appended claims.

### DETAILED DESCRIPTION

It is to be understood the present disclosure is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

Circadian rhythm is a complex process observed in many living organisms, including humans. The 24-hour light-dark cycle of the sun as determined by the orbit and tilt of the earth influences a wide range of biological processes, from photosynthesis in plant life to the sleep-wake cycle of animals (including both diurnal and nocturnal alike). In human subject, the presence of light at a certain wavelength and intensity signal suppression of melatonin production in the suprachiasmatic nucleus and pineal gland, which leads to increased alertness and attention. Contrary to this effect, the absence of intense light allows melatonin production to continue undisturbed. Prior research suggests that blue light above 1000 lux is enough to suppress melatonin production while red light at or under 250 lux provides visual acuity without disrupting normal melatonin production.

Existing devices that emit light into the eyes, typically in the form of glasses, are used for a wide range of environments and situations. On earth, light therapy devices are implemented for a wide range of environments and situations. For instance, light therapy devices may be implemented for night shift work, for seasonal affective disorder, for lack of sunlight during polar seasons, and for various sleep disorders. In space, where light and dark are not provided in the consistent 24-hour cycle, artificial solutions are being used. For example, the International Space Station (ISS) implements a solid-state lighting system that relies on LEDs to emit blue-enriched light for morning and blue-deprived light for pre-sleep. While this system is moderately effective, astronauts still report using caffeine and sleep medications to combat sleep onset delay at night and sleep inertia in the morning. Lack of sleep quantity and quality has the potential to greatly affect performance and mood, especially when presented with tasks requiring large amounts of focus and attention. Negating some of the effects of sleep inertia could, however, potentially reduce drowsiness and improve reaction times in the morning. The benefits of such a device include less reliance on sleep medication as well as greater consistency and adherence to a healthy sleep schedule. These effects would provide a safer and more well-rested atmosphere for space travel.

In order to minimize exposure to blue light in the evenings and its detrimental effects on sleep behavior, various techniques have been put in place including reducing digital screen time before sleep, turning on low blue light/dimming settings on devices included in device operating systems, installing blue light filtering apps on devices, and use of blue light blocking glasses. Blue light blocker glasses have filters or surface coatings in their lenses that block or absorb a portion of emitted blue light, and in some cases UV light. Numerous studies have suggested that if these glasses are used by an individual while working on a digital screen, especially after dark/before bed, they can help reduce exposure to blue light waves that can keep you awake, though more evaluations appear to be needed to quantify the true effects of using blue light blocker glasses. There are different types of blue light blocking glasses available within the commercial market that block different amounts of blue light. The majority of blue light blocking glasses are called “computer glasses” which have clear lenses with a blue tint, which block at a maximum ~ 40% of blue light rays. Their primary application is for individuals working in front of screens all day to minimize eye strain. 100% blue light blocking glasses, also known as “amber glasses”, have red lenses which block nearly 100% of the blue/green/violet light being admitted from screens and lights in our homes. Amber glasses are specifically designed to increase melatonin levels, unlike computer glasses, which are used to alleviate eye strain.

Even though further research is needed to bear out the link between blue blocker glasses and their effect on promoting sleep, both computer and amber glasses are becoming increasingly popular today for a range of applications (e.g., nighttime LED/device work, sleep promotion, addressing flight induced jet lag). These glasses are available through a range of manufacturers, come in a variety of styles (dedicated glasses, clip on glasses), at generally low-price points (e.g., $50-$150/pair).

The present disclosure relates to a continued effort to develop effective blue light blocking technology. Certain embodiments described herein relate to a conceptual dual-mode light therapy system designed to both increase the beneficial effects of blue light exposure in the morning and provide blue light mitigation in the evenings. Various embodiments of a personal light therapy device (e.g., light therapy glasses) disclosed herein have twofold benefits — 1) bright, blue light, which stimulates arousal (when needed upon wakening) and promotes staying awake, and 2) diminished, red light combined with a blue wavelength blocking filter for use before sleep to allow natural sleep onset and to maintain sleep, as necessary. The disclosed light therapy glasses may encourage circadian rhythm synchronization with a normal 24-hour sleep-wake cycle even with inconsistent exposure to natural light. The disclosed light therapy glasses may provide both blue light therapy above 1000 lux (e.g., about 1067 lux with a wavelength of 468 nm) in the morning and red light therapy at or under 250 lux (e.g., about 249 lux with a wavelength of 632 nm) in the evening. In various embodiments, the inclusion of a red or amber filter that blocks ambient blue light gives the added benefit of blocking melatonin suppressing blue wavelengths before sleep. The combined effects will promote, without the use of medicine, faster sleep onset and reduced sleep inertia in astronauts or other users that may benefit from such personalized light therapy. Examples of other users include, but are not limited to, transoceanic flight travelers, military users, truck drivers (such as long haul truck drivers), and other users with occupations that need to stay awake and/or sleep in prescribed ways.

In various embodiments, a personal light therapy device provides a user with bright, blue light therapy above 1000 lux in the morning and low light, red-light therapy (e.g., red light combined with a blue light blocking filter) at or under 250 lux in the evening to encourage synchronization with a normal 24-hour sleep-wake cycle/circadian rhythm. In certain embodiments, the personal light therapy device is designed and constructed as a pair of glasses that combine both light therapy capabilities. Blue light at a wavelength of ~ 468 nm may be used to induce alertness/stimulate arousal in the morning (e.g., by suppression of melatonin production), and red light at a wavelength of ~632 nm may be used to block blue light in the evenings to compensate for the blue light’s suppressive properties and promote melatonin production and better sleep.

In some embodiments, the device operates through use of a push operated tactile switch; one on either side of the frames, which controls the two-color modes of light. The red-light mode is achieved by use of a red-colored removable filter constructed of a transparent sheet providing blue light filtering. A wireless charger is used to recharge the 3.7 V/350 mAh lithium-ion polymer battery when the glasses are not in use, and an adjustable head strap is attached to the glasses to allow their use in microgravity situations (e.g., space environment). In one embodiment, eight, 3 mm diffused blue LED and eight, 3 mm diffused red LED are used to provide appropriate lighting and are housed in the frames along with sensors and computer controllers.

Embodiments of light therapy glasses may include a combination of technologies: 1) emittance of blue light at specific wavelengths and intensities that promote alertness; 2) emittance of red light at specific wavelengths and intensities that promote drowsiness; and 3) attachment of a filter to the frames that removes all blue wavelengths of light from vision, to expedite drowsiness when needed. In some example embodiments, the light therapy glasses are implemented in a microgravity environment such as those experienced by astronauts.

As described above, in various embodiments, light therapy glasses as disclosed herein may help an individual regulate their circadian rhythm. For instance, implementation of the optimal intensities and wavelengths of blue light may be implemented to induce alertness in the morning and red light may be implemented to prevent melatonin disruption before sleep. In some embodiments, a red colored, removable filter prevents exposure to blue wavelengths of light in the evening. In certain embodiments, the light therapy glasses include a push switch on either side of the frames of the glasses to control the two color modes of light. In a first color mode of light, blue light of 1000 lux at approximately 468 nm wavelength is emitted. In a second color mode of light, red light of 250 lux at approximately 632 nm is emitted. In some embodiments, a wireless charger is implemented to recharge the battery of the glasses when not in use. Additionally, an adjustable head strap may be implemented for security in microgravity.

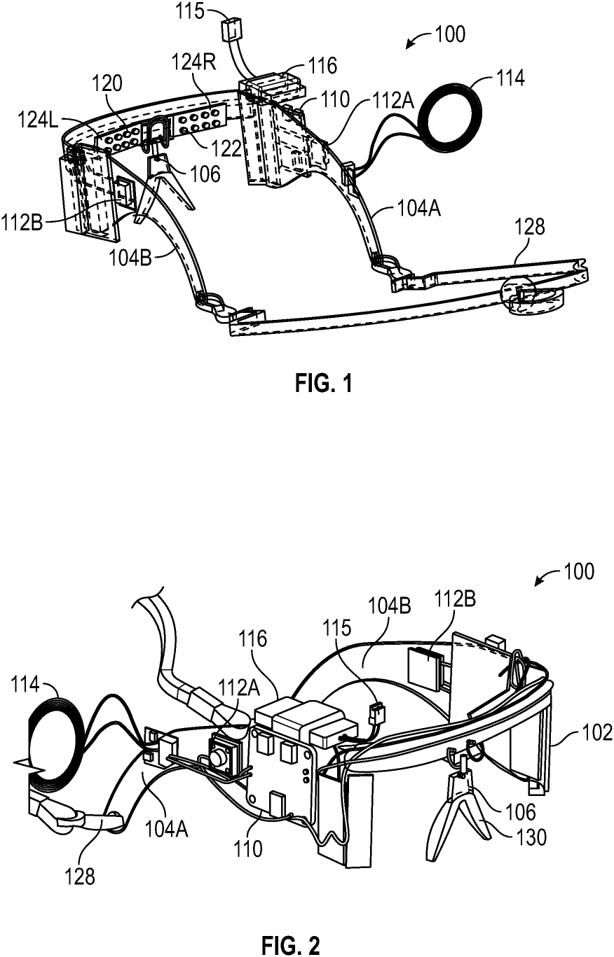

FIG. 1 depicts a rear and left perspective view of light therapy glasses 100, according to some embodiments. FIG. 2 depicts a front and right perspective view of light therapy glasses 100, according to some embodiments. In the illustrated embodiments, glasses 100 include frames 102. Frames 102 may include earpieces 104 (e.g., right earpiece 104A and left earpiece 104B) and nose piece 106. In some embodiments, nose piece bridge 130 is coupled to nose piece 106. Nose piece bridge 130 may be, for example, an OnGuard node piece bridge or other structure for secure engagement with a user’s nose. In some embodiments, lanyard 128 is coupled to earpieces 104. Lanyard 128 may be an adjustable lanyard head strap attached to earpieces 104 to avoid allowing glasses to inadvertently come off a user’s head.

As shown in FIG. 1, glasses 100 include blue light emitting diodes (LEDs) 120 and red LEDs 122 attached to circuit boards 124. Circuit boards 124 may be, for example, double-sided printed circuit boards (PCBs). In certain embodiments, blue LEDs 120 are diffused blue, 3 mm diameter LEDs that provide light at a wavelength of 468 nm and red LEDs 122 are diffused red, 3 mm diameter LEDs that provide light at a wavelength of 632 nm. In various embodiments, circuit boards 124 may include a first circuit board 124L positioned above where a user’s left eye would be and a second circuit board 124R positioned above where a user’s right eye would be when glasses are worn by the user. Thus, the user, when wearing glasses 100, has a set of blue LEDs 120 and a set of red LEDs 122 positioned above each eye.

In certain embodiments, circuit boards 124 are attached to a periphery of frames 102. Thus, blue LEDs 120 and red LEDs 122 are positioned on the periphery of frames 102. Positioning the LEDs on the periphery of frames 102 places the LEDs where they will not block the vision of the user when wearing glasses 100. In various embodiments, the LEDs are positioned in a superior position to the user’s eyes (e.g., in a position just above the user’s field of view when wearing glasses 100). Accordingly, the LEDs may be positioned to not provide light directly into the user’s eyes (e.g., have direct line-of-sight to the user’s irises) when the LEDs are turned on. In some embodiments, blue LEDs 120 and red LEDs 122 are positioned a specified distance from the user’s eyes on the periphery of frames 102 (and in the superior position) when the user wears glasses 100. The specified distance may, in certain embodiments, be a distance that that allows the light from the LEDs to be picked up by the eyes while not blinding or distracting the user (e.g., the user encounters the LED light as indirect or “ambient” light while wearing glasses 100). The specified distance may, in some instances, be dependent on the angle of position between the user’s eyes and the LEDs (e.g., the angle between the superior position of the LEDs on the periphery of frames 102 and the user’s eyes when the glasses 100 are worn by the user). With the LEDs positioned on the periphery of frames 102, glasses 100 may provide blue light or red light at close distances, which produces effective melatonin suppression (via blue light) or melatonin promotion (via red light), while not distracting the user from any tasks being performed while wearing the glasses (e.g., piloting while receiving blue light or reading while receiving red light).Placing blue light nearer a user’s eye (e.g., on the glasses) provides more benefits than ambient blue light or other blue light sources not placed on a user’s head. For instance, placing blue light close to a user’s eyes while wearing glasses has been shown to be more effective in reducing melatonin production by the user than when blue light is ambient to the user. Additionally, placing blue light with at a wavelength of 468 nm and a brightness above 1000 lux has also been shown to be effective at melatonin suppression, thereby promoting alertness in the user. For red light, placing red light at a wavelength of 632 nm with a brightness of about 250 lux has been shown to promote melatonin production.

As shown in FIGS. 1 and 2, glasses 100 further include controller 110, switches 112, and power source 116 coupled to frames 102. In certain embodiments, power source 116 is a rechargeable battery. For example, power source 116 may be a lithium ion battery. In one example embodiment, power source 116 is a 3.7 V 350 mAh lithium ion polymer battery available from Adafruit Industries LLC (New York, NY). Power source 116 may be charged through either wired charging port 115 or wireless charging device 114. Wired charging port 115 may be, for example, a micro-LiPo charger with a micro-USB port. Wireless charging device 114 may be, for example, an inductive charger such as a universal Qi wireless charging transmitter and inductive charging set coil.

In various embodiments, controller 110 is a circuit board controller that is capable of receiving power from power source 116 and distributing (e.g., providing) power to LEDs 120, 122. In one example embodiment, controller 110 is a nano circuit board controller available from Arduino® (Turin, Italy). In certain embodiments, switches 112 are coupled to controller 110 to determine which LEDs (e.g., LEDs 120 or LEDs 122) receive power from the controller. For instance, in the illustrated embodiment, switch 112A is utilized to turn on/off power to blue LEDs 120 and switch 112B is utilized to turn on/off power to red LEDs 122. Switches 112 may be, for example, tactile button switches. In some embodiments, controller 110 and switches 112 may operate to inhibit both sets of LEDs being turned on together. For example, controller 110 and/or switches may be programmed to inhibit blue light LEDs 120 and red light LEDs 122 from being turned on at the same time. Accordingly, red light LEDs 122 cannot be turned on when blue light LEDs 120 are turned on, and vice versa. In some contemplated embodiments, a single switch 112 may be used for both sets of LEDs 120, 122. For instance, a selector switch may be utilized with one position for blue LEDs 120 on, one position for red LEDs 122 on, and one position for all LEDs off.

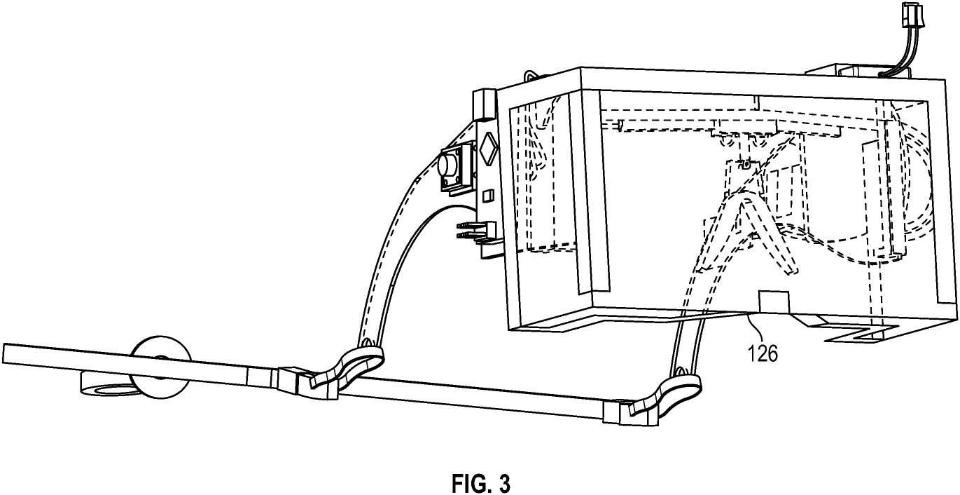

In various embodiments, a red filter (e.g., a filter that blocks ambient blue light) is removably coupled to glasses 100. FIG. 3 depicts a front perspective view of glasses 100 with a filter sheet attached to the glasses, according to some embodiments. In the illustrated embodiment, sheet 126 is attached to frames 102 of glasses 100 to place the sheet in the field of view of the user’s eyes when the glasses are worn by the user. In certain embodiments, sheet 126 is an amber blue-light filtering transparent sheet. In certain embodiments, sheet 126 is removably coupled to frames 102. For instance, sheet 126 may have attachment mechanisms that allow the sheet to be attached/removed from frames 102 as desired by the user. Some embodiments may be contemplated where sheet 126 is swung in/out of the field of view of the user’s eyes. For instance, sheet 126 may be attached to frames 102 with a hinged connector to allow the sheet to be swiveled up/down. Various other mechanisms may also be contemplated that server to allow the user (or another person) to move sheet 126 in/out of the field of view user as needed so that the user has the sheet in position when getting ready for sleep (e.g. when the red LEDs 122 are illuminated) and the sheet is out of the user’s field of view when attempting to stay alert (e.g., when the blue LEDs 120 are illuminated).

With the transparent sheet 126, the user can see through sheet while blue light is inhibited (e.g., filtered) from reaching the user’s eyes. Accordingly, the user can continue to view through glasses 100 (e.g., read) while wearing the glasses and begin to wind down and get ready for sleep. The combination of red light LEDs 122 and sheet 126 may produce more effective sleep readiness for the wearer of glasses 100. For instance, blocking the ambient blue light with sheet 126 will inhibit blue light suppression of melatonin production that is simultaneously being promoted by illumination of red LEDs 122 on glasses 100. Thus, glasses 100, when used for sleep readiness, may provide enhanced effectiveness in readying the user for sleep over simple blue light deprivation since melatonin production is promoted with the red LED light and ambient blue light that may inhibit melatonin production is inhibited from being received by the user using a filter that is in close proximity to the user’s eyes (where some methods may shine blue deprived light from larger distances).

Various embodiments of the light therapy glasses 100 described herein may have the following advantages:
Light therapy glasses 100 provide two modes of operation in a single pair of glasses associated with sleep cycles for a user - a first mode includes blue light stimulation for alertness and a second mode includes red light therapy for melatonin production in combination with ambient blue light inhibiting capabilities through use of a removable filter. Thus, a single pair of glasses functions for both promoting alertness and readying a user for sleep.
Blue light stimulation is provided at a wavelength and brightness that best promotes alertness of the user. Blue light is also provided at distances that promote higher benefits of the blue light for reducing melatonin production and promoting alertness.
Use of a single device to provide both blue light/redlight capacities alleviates the need for the user to swap between two separate light therapy devices during different periods of use.
Blue and red lights are placed at positions along periphery of frame to inhibit direct line of sight light incident on the user’s eyes, reducing distraction to the user and preventing unwanted blinding of the user.
Dual mode device activation is realized through use of one or more easy to operate integrated, push button switches coupled to a single controller and a single power source.

Various measurement tests may be conducted to test light therapy gases include, but are not limited to:
Heart Rate Variability (HRV) - HRV is an objective measurement of the variability in time intervals between consecutive heartbeats. This measurement is strongly correlated with the ability to respond to stressful or varied stimuli, and is most variable during sleep or pre-sleep and least variable when awake. HRV oscillations of the heart are complex and nonlinear which reflects regulation of functions such as blood pressure, gas exchange, and autonomic balance. Participants will be using a chest-strap HR monitor connected to the Elite HRV app, which will provide a snapshot of HRV during data collection.
Electroencephalography (EEG) - EEG provides a continuous readout of brain wave activity, which can be interpreted according to wave types. Various EEG studies have shown associations between alertness, alpha waves, theta waves, and different performance measurements. These wave types indicate alertness and attention. The EEG will be applied and data recorded for morning and evening testing sessions in order to compare the change in brain wave activity.
Psychomotor Vigilance Test (PVT) - The PVT was developed by NASA in order to assess reaction times. PVT is an accurate way of measuring performance impairment that arises from sleep loss and circadian desynchronization. This test has a duration of 5 minutes and will be administered several times during the morning and evening testing sessions. This test allows us to objectively measure the difference in reaction times before and after light therapy intervention.
Subjective Measurements - The Toronto Hospital Alertness Test is a scale which allows participants to record their subjective interpretation of 1 week’s worth of sleep and fatigue. This test will be administered after 1 week of control sleep conditions, and after the following week of light therapy intervention. This will allow us to compare the participant’s feelings of sleep and fatigue before and after light therapy longitudinally. Additionally, the Karolinska Sleepiness scale and a sleep diary log will give participants a chance to record sleep data every day for more short term comparison.

Any examples included are to demonstrate preferred embodiments of the present disclosure. It should be appreciated by those of skill in the art that the techniques disclosed in the examples which follow represent techniques discovered by the inventor to function well in the practice of the disclosed embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

Further modifications and alternative embodiments of various aspects of the present disclosure will be apparent to those skilled in the art in view of this description. Accordingly, this description is to be construed as illustrative only and is for the purpose of teaching those skilled in the art the general manner of carrying out the disclosed embodiments. It is to be understood that the forms of the embodiments shown and described herein are to be taken as examples of embodiments. Elements and materials may be substituted for those illustrated and described herein, parts and processes may be reversed, and certain features of the present disclosure may be utilized independently, all as would be apparent to one skilled in the art after having the benefit of this description of the present disclosure. Changes may be made in the elements described herein without departing from the spirit and scope of the present disclosure as described in the following claims.

What is claimed is:

1. A light therapy device, comprising:
eyeglasses frames;
a blue-light filtering transparent sheet coupled to the frames, wherein the blue-light filtering transparent sheet is configured to be moved in and out of a field of view of both eyes of a wearer of the frames;
a set of blue light LEDs positioned on a periphery of the frames and configured to direct blue light towards both eyes of the wearer of the frames;
a set of red light LEDs positioned on the periphery of the frames and configured to direct red light towards both eyes of the wearer of the frames;
a power source coupled to the frames;
a controller coupled to the frames, the controller being electrically coupled to the power source, the blue light LEDs, and the red light LEDs, the controller being configured to control distribution of electrical power from the power source to the blue light LEDs and the red light LEDs; and
at least one switch coupled to the frames and the controller, wherein the at least one switch is operable to determine whether the electrical power is distributed to the blue light LEDs or the red light LEDs by the controller.

2. The light therapy device of claim 1, wherein the electrical power is distributed to the blue light LEDs and the blue light LEDs are turned on during periods of time the wearer is supposed to stay awake.

3. The light therapy device of claim 1, wherein the electrical power is distributed to the red light LEDs and the red light LEDs are turned on during periods of time the wearer is supposed to be falling asleep or asleep.

4. The light therapy device of claim 3, wherein the blue-light filtering transparent sheet is configured to be positioned in the field of view of both eyes of the wearer when the red light LEDs are turned on.

5. The light therapy device of claim 1, wherein the blue light LEDs and the red light LEDs are positioned on periphery of the frames such that the LEDs are not directly in the field of view of the wearer’s eyes.

6. The light therapy device of claim 1, wherein the blue light LEDs and the red light LEDs are positioned on the periphery of the frames a specified distance from the wearer’s eyes.

7. The light therapy device of claim 6, wherein the specified distance is a distance at which the wearer’s eyes can detect light from the LEDs.

8. The light therapy device of claim 1, wherein the blue light LEDs are configured to output blue light at an illumination of at least 1000 lux at a wavelength of 468 nm.

9. The light therapy device of claim 1, wherein the red light LEDs are configured to output red light at an illumination of about 250 lux at a wavelength of 632 nm.

10. The light therapy device of claim 1, wherein the at least one switch includes a first user switch and a second user switch, wherein the first user switch is configured to turn on/off the blue LED lights, and wherein the second user switch is configured to turn on/off the red LED lights.

11. The light therapy device of claim 1, wherein the at least one switch and the controller are configured to inhibit the red light LEDs from being turned on when the blue light LEDs are turned on and inhibit the blue light LEDs from being turned on when the red light LEDs are turned on.

12. The light therapy device of claim 1, wherein the power source is a rechargeable battery coupled to the controller.

13. The light therapy device of claim 12, wherein the rechargeable battery includes a wired charging port.

14. The light therapy device of claim 12, further comprising a wireless charging device coupled to the rechargeable battery.

15. The light therapy device of claim 14, wherein the wireless charging device is an inductive charging device.

16. The light therapy device of claim 1, wherein the sheet is removably coupled to the frames, the sheet being positioned in the field of view of both eyes of the wearer when coupled to the frames.

17. The light therapy device of claim 1, wherein the sheet is an amber, blue-light filtering transparent sheet.

18. A method for promoting a user to stay awake or fall asleep, comprising:
placing eyeglasses frames on the user’s head, wherein the eyeglass frames includes both: a set of blue light LEDs positioned on a periphery of the frames that direct blue
light towards both eyes of the user; and
a set of red light LEDs positioned on the periphery of the frames that direct red light towards both eyes of the wearer of the frames;
turning on the blue light LEDs during periods of time in which the user is supposed to remain awake; and
turning on the red light LEDs during periods of time in which the user is supposed to fall asleep or be asleep.

19. The method of claim 18, further comprising placing a blue-light filtering transparent sheet in a field of view of the user’s eyes when the red light LEDs are turned on, wherein the blue-light filtering transparent sheet is coupled to the frames.

20. The method of claim 18, wherein the red light LEDs are turned off when the blue light LEDs are turned on, and wherein the blue light LEDs are turned off when the red light LEDs are turned on.
